(19) United States
(12) Patent Application Publication
(10) Pub. No.: US 2017/0013795 A1
(43) Pub. Date: Jan. 19, 2017

(54) METHODS AND SYSTEMS FOR EXTRACTING DICOT EMBRYOS

(71) Applicant: PIONEER HI-BRED INTERNATIONAL, INC., JOHNSTON, IA (US)

(72) Inventors: CLIFFORD P. HUNTER, MILILANI, HI (US); JEFFREY DALE WILLE, ANKENY, IA (US)

(73) Assignee: PIONEER HI-BRED INTERNATIONAL, INC., JOHNSTON, IA (US)

(21) Appl. No.: 15/123,971

(22) PCT Filed: Mar. 9, 2015
(86) PCT No.: PCT/US15/19444

§ 371 Date: Sep. 6, 2016

Related U.S. Application Data
(60) Provisional application No. 61/949,738, filed on Mar. 7, 2014.

(51) Int. Cl. A01H 4/00; G01N 35/00; G01N 1/08; C12Q 1/68; C12N 5/04

(52) U.S. Cl. CPC A01H 4/008; C12Q 1/6895; C12N 5/04; G01N 1/08; G01N 35/00; C12N 2501/00; G01N 2001/045

(57) ABSTRACT

Methods and systems are provided which permit extraction of a dicot embryo of a dicot seed without damage to the dicot embryo. Methods disclosed herein provide embryos for uses in plant breeding and research procedures.

### FIELD

The present invention relates generally to systems and methods for extracting dicot embryos from dicot seeds without damaging the dicot embryos.

### BACKGROUND

Various large-scale systems have been proposed for extracting dicot embryos; however, these systems have generally performed in a manner that damages the dicot embryos during extraction and extracts portions of the dicot seed other than the dicot embryo. Current automated systems and methods for extracting embryos typically extract the embryos through a distal end (crown) of the seeds while the seeds are still on their carrier. For example, a corn embryo is often extracted by forcing the embryo through the crown of a kernel while the kernel is attached to a cob. The anatomy of corn kernels and other seeds often requires application of significant forces to extract embryos through the distal end (crown) of the seeds. These significant forces create undesirable stress on the embryo. Often, current automated embryo extraction methods also require sieving of the extracted material to ensure separation of the embryo from other materials. The sieving step requires application of further force to the embryos, thereby resulting in further stress on the embryos. Thus, there is a need for alternative methods to extract isolated, undamaged dicot embryos from dicot seeds.

### SUMMARY

Disclosed herein are methods and systems which extract a dicot embryo from a singulated immature dicot seed that generally has been removed from its biological carrier, which as is known to those of skill in the art, may be a pod, a cob, or other plant structure associated with a seed. Herein, it may be referred to as a biological carrier to distinguish it from a part of a device that may carry objects. Those of skill in the art can distinguish the usage of the term. An immature dicot seed can be obtained and may then placed in a liquid bath to achieve a desired orientation. In general, methods may comprise a force applied to the dicot seed to extract the dicot embryo. Optionally, the dicot embryo may be released into the liquid bath.

Methods and systems disclosed herein may comprise automatically isolating a dicot embryo from an immature dicot seed having a proximal end and an opposed distal end. For orientation and understanding, prior to removal of the immature dicot seed from a biological carrier, the proximal end of the immature dicot seed is attached to the biological carrier and the distal end of the immature dicot seed is spaced from the biological carrier. An isolated immature dicot seed having an opening in the proximal end of the dicot seed can be provided following removal of the immature dicot seed from the biological carrier. At least a portion of the proximal end of the dicot seed may be removed to form the opening in the proximal end of the dicot seed without damage to the dicot embryo. The dicot embryo can be extracted through the opening in the proximal end of the dicot seed, and the dicot embryo is generally undamaged following extraction. Following extraction of the dicot embryo, the undamaged dicot embryo may retain an ability to grow with full viability and vigor.

Methods and systems disclosed herein may comprise regenerating a plant from a dicot embryo of an immature dicot seed. The dicot embryo can be automatically extracted from the immature dicot seed without damage to the dicot embryo. The dicot embryo can have a meristematic section and a cotyledon section. A sample portion of the immature cotyledon section of the dicot embryo can be removed without damage to the extracted dicot embryo. The sample portion and the viable embryo can be tracked and identified in a 1 to 1 relationship. The sample portion of the immature cotyledon section of the dicot embryo can be genetically analyzed, and a plant can be regenerated from the remaining portion of the dicot embryo (including the meristematic section). Methods and systems disclosed herein may permit individual analysis of each of a plurality of immature dicot seeds. A dicot embryo from each respective dicot seed of a plurality of immature dicot seeds can be automatically extracted, and a sample portion of the immature cotyledon section of the dicot embryo of each respective dicot seed can be removed without damage to the dicot embryo. A sample portion of the embryo, such as a immature cotyledon section of a dicot embryo, of each respective dicot seed can be analyzed, for example, genetically analyzed, and the remaining portions of the dicot embryo of each respective dicot seed can be selected or discarded on the basis of the genetic analysis of the sample portion of the immature cotyledon section of the dicot embryo of each respective dicot seed. For example, a sample may be obtained from a portion of the embryo, and may comprise one or more cells. It is contemplated that the sample from the embryo can be as small as a single nucleus. The remaining portions of the dicot embryo(s) of at least one dicot seed of the plurality of dicot seeds can be selected, and at least one plant can be regenerated from the remaining portions of each respective selected dicot embryo.

### BRIEF DESCRIPTION OF THE FIGURES

FIG. 1 is a schematic diagram of an immature seed. As shown, the embryo comprises cotyledon tissue and meristematic tissue.

FIG. 2 is a flow chart depicting an exemplary method of extracting a dicot embryo from an immature dicot seed as disclosed herein.

FIG. 3 is a flow chart depicting another exemplary method of extracting a dicot embryo from an immature dicot seed as disclosed herein.

FIG. 4A is a flow chart depicting an exemplary method of regenerating a plant from portions of a dicot embryo following extraction from an immature dicot seed. FIG. 4B is a flow chart depicting an exemplary method of regenerating a plant from portions of at least one dicot embryo selected among a plurality of dicot embryos following extraction of the dicot embryos from immature dicot seeds. FIG. 4C is a flow chart depicting an exemplary method of tracking the location of at least one extracted dicot embryo during the method depicted in FIG. 4B. Tracking may comprise identifying and relating an embryo, a sample of a particular embryo or or portion of a seed to the original seed. In general, the identity and relation of a seed to its geographic location of growth or genetic sources (parentage) are known or tracked.

FIG. 5A is a perspective view of an exemplary system for performing one or more of the dicot embryo extraction methods disclosed herein. FIG. 5B is a schematic diagram depicting the mechanics of an exemplary holding fixture for use in the system of FIG. 5A.

FIG. 6A is a top perspective view of another exemplary system for performing one or more of the dicot embryo extraction methods disclosed herein. FIG. 6B is a side perspective view of the system of FIG. 6A. FIG. 6C is a close-up perspective view of an exemplary extraction subassembly of the system of FIGS. 6A-6B.

FIG. 7 is a perspective view of an exemplary gripping and extraction apparatus as disclosed herein.

FIGS. 8A-8B provide a series of images depicting exemplary semi-automated experimental methods of extracting embryos using an extraction device as disclosed herein.

FIG. 9 is a table showing a comparison of the condition of corn embryos extracted using various experimental methods, as further described herein.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a”, “an”, and “the” include plural referents unless the context clearly dictates otherwise. Thus, for example, reference to “a sample portion” can include a plurality of such sample portions, and so forth. All technical and scientific terms used herein have the same meaning as commonly understood to one of ordinary skill in the art to which this invention belongs unless clearly indicated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

### I. Overview

The present invention comprises methods and systems for extracting the dicot embryos of isolated immature dicot seeds. It is contemplated that disclosed methods and systems can be used to extract the dicot embryo of an individual dicot seed without damaging the dicot embryo. In exemplary aspects, it is contemplated that systems and methods disclosed herein can accomplish extraction of sunflower embryos using about 20% of the time required to extract sunflower embryos using traditional excision methods cutting through the side of a kernel. It is further contemplated that the embryos extracted using the systems and methods disclosed herein can be sufficiently separated from other materials that sieving and other mechanical embryo separation steps are not required.

In operation, the systems and methods disclosed herein can permit extraction of embryos from dicot seeds on a seed-by-seed basis. It is contemplated that the seed-by-seed approaches disclosed herein can advantageously permit improved cleaning and sterilization of individual seeds, for example, allowing sterilization of the entire surface of the seed, compared to batch processes. It is contemplated that disclosed methods and systems can permit tracking and/or analysis of individual dicot embryos following extraction of the dicot embryos from respective dicot seeds. It is contemplated that disclosed methods and systems can permit the regeneration of a plant using an individual extracted dicot embryo.

Exemplary methods of extracting a dicot embryo after orienting a dicot seed within a liquid bath, automatically extracting dicot embryos, and regenerating a plant from an immature dicot seed are disclosed. Unless otherwise stated, it is contemplated that, although these exemplary methods are described separately, the steps of any one of the disclosed methods can be used in combination with the steps of any of the other disclosed method to arrive at a method of extracting a dicot embryo from an immature dicot seed. Thus, it is contemplated that one or more steps of a disclosed method of extracting a dicot embryo after orienting a dicot seed within a liquid bath can be used in combination with one or more steps of the disclosed methods of automatically extracting dicot embryos and/or regenerating a plant from an immature dicot seed. Similarly, it is contemplated that one or more steps of disclosed methods of automatically extracting dicot embryos can be used in combination with one or more steps of disclosed methods of extracting a dicot embryo after orienting a dicot seed within a liquid bath and/or regenerating a plant from an immature dicot seed. It is further contemplated that one or more steps of disclosed methods of regenerating a plant can be used in combination with one or more steps of disclosed methods of extracting a dicot embryo after orienting a dicot seed within a liquid bath and/or automatically isolating dicot embryos. It is further contemplated that, unless otherwise stated, any of the steps of disclosed embryo extraction methods can be performed in an automated fashion.

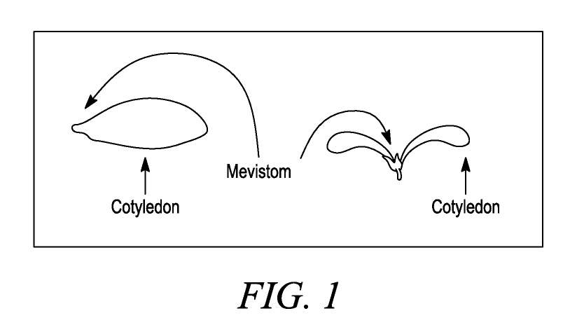

FIG. 1 depicts various components of a seed, including the embryo. As shown in FIG. 1, the embryo is encased by the pericarp.

As used herein, and as shown in FIG. 1, “embryo” or “dicot embryo” consists of a meristematic section and cotyledon section.

As used herein, the “meristematic section” of the dicot embryo refers to the essential genetic information and embryonic structures required for the dicot embryo to grow into a plant.

As used herein, the “cotyledon section” of the dicot embryo refers to the tissue that generally surrounds the meristematic section within the dicot embryo and stores nutrients mobilized during germination of a dicot seed. As used herein, an “immature” dicot seed refers to a dicot seed harvested within a selected number of days after pollination. As used herein, an “immature” dicot seed refers to a dicot seed harvested after fertilization but prior to physiological maturity and, except in the case of hard coat seeds, does not germinate in water without external nutrients. In examples, the harvest is contemplated to occur between 200 and 750 Growing Degree Days after pollination.

As used herein, an “undamaged” embryo or an embryo “without damage” refers to an embryo that does not exhibit substantial bruising after being extracted and retains an ability to grow with full viability and vigor. For example, a dicot embryo is “undamaged” following extraction if the viability and vigor of the dicot embryo is substantially unchanged following extraction. In exemplary aspects, it is contemplated that a dicot embryo is “undamaged” following extraction if the viability and vigor of the dicot embryo are at least 50% of the viability and vigor of dicot embryos extracted using conventional hand extraction methods. In one exemplary aspect, a dicot embryo is “undamaged” following extraction if the viability and vigor of the dicot embryo are at least 60% of the viability and vigor of dicot embryos extracted using conventional hand extraction methods. In another exemplary aspect, a dicot embryo is “undamaged” following extraction if the viability and vigor of the dicot embryo are at least 70% of the viability and vigor of dicot embryos extracted using conventional hand extraction methods. In another exemplary aspect, a dicot embryo is “undamaged” following extraction if the viability and vigor of the dicot embryo are at least 80% of the viability and vigor of dicot embryos extracted using conventional hand extraction methods. In still another exemplary aspect, a dicot embryo is “undamaged” following extraction if the viability and vigor of the dicot embryo are at least 90% of the viability and vigor of dicot embryos extracted using conventional hand extraction methods. In a further exemplary aspect, a dicot embryo is “undamaged” following extraction if the viability and vigor of the dicot embryo are at least 95% of the viability and vigor of dicot embryos extracted using conventional hand extraction methods. In general, viability refers to the ability of the embryo to germinate and develop into a plantlet under tissue culture or growth medium conditions known to those skilled in the art. In general, vigor refers to the growth and development of the resultant plantlet, up to and including male and female flowering, kernel development, reached kernel physiological maturity.

] In other exemplary aspects, it is contemplated that a dicot embryo is “undamaged” following extraction if the germination of the dicot embryo generates a viable plantelet.

As used herein, the term “automatic” or “automatically” refers to the use of mechanical, electrical, software, imaging, vision-based and/or other known automation-based technologies to augment processes typically performed by human interaction.

II. Methods of Extracting Dicot Embryos from Immature Dicot Seeds

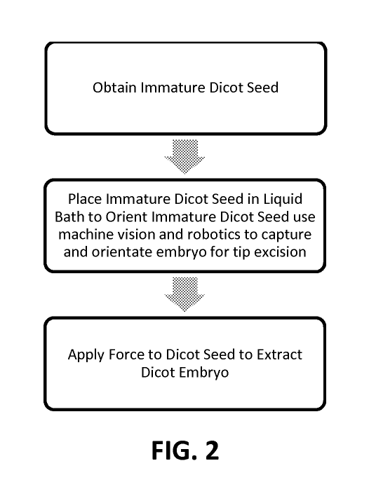

In various aspects, and with reference to FIG. 2, a method of extracting a dicot embryo from an immature dicot seed is provided. In these aspects, a method can comprise obtaining an immature dicot seed. Optionally, in one aspect, a method can further comprise placing the immature dicot seed in a liquid bath. In an aspect, it is contemplated that placement of the immature dicot seed can cause the immature dicot seed to orient itself in a desired orientation. For example, it is contemplated that positioning of an immature sunflower seed within a liquid bath can cause the sunflower seed to orient itself with both distal and proximal ends of the seed facing at the surface of the liquid bath as the bow and stern of a boat In an aspect, a method can further comprise applying force to extract the dicot embryo from the immature dicot seed. Optionally, when the immature dicot seed is positioned in a liquid bath, the force can be applied such that the dicot embryo is released into the liquid bath. In an aspect, it is contemplated that the dicot seed can optionally be wholly submerged in the liquid bath during the step of applying force to the dicot embryo. Alternatively, it is contemplated that the distal end of the dicot seed can be submerged in the liquid bath while at least a portion of the proximal end is positioned above the surface of the liquid bath. Optionally, it is contemplated that the entire dicot seed can be positioned above the surface of the liquid bath such that the dicot embryo is released into the liquid bath upon extraction. Optionally, in an aspect, it is contemplated that an immature dicot seed can be an immature sunflower seed.

In further aspects, it is contemplated that a dicot embryo can be undamaged following extraction from the dicot seed. In these aspects, it is contemplated that, following extraction of a dicot embryo from the immature dicot seed, the dicot embryo can retain an ability to grow with full viability and vigor. More generally, it is contemplated that the dicot embryo can retain the ability to germinate following extraction from the dicot seed. Optionally, in additional aspects, it is contemplated that a dicot embryo of the dicot seed can be extracted such that other material of the dicot seed is not substantially extracted from the dicot seed. It is still further contemplated that an extracted dicot embryo of a dicot seed can be sufficiently separated from other material following extraction such that sieving of the extracted dicot embryo is not required.

In an aspect, the immature dicot seed can have a proximal end and an opposed distal end. In an aspect, it is contemplated that, prior to removal of the immature dicot seed from a biological carrier, the proximal end of the immature dicot seed can be attached to the biological carrier and the distal end of the immature dicot seed can be spaced from the biological carrier. In exemplary aspects, when the immature dicot seed is an immature sunflower seed, the proximal end of the sunflower seed can comprise a tip cap of the sunflower seed and the distal end of the sunflower seed can comprise a crown of the sunflower seed.

Optionally, in some aspects, it is contemplated that an opening or hole can be formed in the proximal end of the dicot seed. In an aspect, an opening or hole in the proximal end of the dicot seed can optionally be formed before force is applied to the dicot seed. In an aspect, it is contemplated that the dicot seed can be positioned in any location permitting application of force to the dicot seed as disclosed herein. In aspects, it is contemplated that an opening or hole in the proximal end of the dicot seed can optionally be formed before the dicot seed is obtained. Alternatively, in other optional aspects, it is contemplated that an opening or hole in the proximal end of the dicot seed can be made after the dicot seed has been obtained. Optionally, in some aspects, it is contemplated that the method can further comprise cleaning at least the proximal end of the dicot seed. In aspects, a step of cleaning at least the proximal end of the dicot seed can comprise sterilizing the surface of the dicot seed. In other exemplary aspects, it is contemplated that a dicot seed can be cleaned before the dicot seed is obtained. Optionally, in aspects, the surface of the dicot seed can be sterilized before the dicot seed is obtained.

Optionally, in other aspects, it is contemplated that the dicot embryo can be extracted from the dicot seed without the need for forming an opening or hole in the dicot seed. In these aspects, it is contemplated that at least a portion of the dicot seed can be placed in the liquid bath. Optionally, it is contemplated that the dicot seed can optionally be wholly submerged in the liquid bath prior to the application of force to the dicot seed. Alternatively, it is contemplated that the distal end of the dicot seed can optionally be submerged in the liquid bath while at least a portion of the proximal end is positioned above the surface of the liquid bath. It is further contemplated that the entire dicot seed can be positioned above the surface of the liquid bath prior to and during the application of force to the dicot seed.

In exemplary aspects, when an opening or hole is formed in the dicot seed, it is contemplated that the step of applying force to extract the dicot embryo can comprise applying pressure to at least one side portion of the dicot seed to force the dicot embryo through the hole in the proximal end of the dicot seed. In these aspects, the dicot seed comprises side portions extending from the proximal end to the distal end of the seed. It is contemplated that the step of applying pressure to at least one side portion of the dicot seed can comprise supporting the dicot seed in a selected orientation while pressure is applied to the at least one side portion of the dicot seed. In an exemplary aspect, the step of applying pressure to at least one side portion of the dicot seed can comprise applying a rolling force to the at least one side portion moving in a direction from the distal end of the seed toward the proximal end of the seed. In other exemplary aspects, when an opening or hole is not formed in the dicot seed, it is contemplated that the step of applying force to extract the dicot embryo can comprise applying pressure to the at least one side portion of the dicot seed to split a portion of the proximal end of the dicot seed and to force the dicot embryo through defined by the split portion of the dicot seed. In these aspects, it is contemplated that the step of applying pressure to the at least one side portion of the dicot seed can comprise supporting the dicot seed in a selected orientation while pressure is applied to the at least one side portion of the dicot seed.

Optionally, in some aspects, the dicot seed can be wholly submerged in the liquid bath when the force is applied to the dicot seed. In these aspects, it is contemplated that the selected orientation of the dicot seed can correspond to the proximal end of the dicot seed being pointed toward the surface of the liquid bath. In other aspects, it is contemplated that the dicot seed can be only partially submerged in the liquid bath when the force is applied to the dicot seed. In these aspects, it is contemplated that the selected orientation of the dicot seed can correspond to the distal end of the dicot seed being submerged in the liquid bath while at least a portion of the proximal end of the dicot seed is positioned above the surface of the liquid bath. In still other aspects, the entire dicot seed can be positioned above the liquid bath.

In exemplary aspects, it is contemplated that a dicot seed can be positioned in the selected orientation by positioning the dicot seed in the liquid bath and permitting the dicot seed to orient in the liquid bath such that the proximal end of the dicot seed points upwardly (toward the surface of the liquid bath). The distal end of the seeds can be painted with glue containing weighted particles to cause the distal end to sink. In these aspects, it is contemplated that dicot seeds having excess material (e.g., “bee's wings” or cob material), impurities, or trapped air may not orient as desired. In order to address the issue of trapped air, it is contemplated that the methods as disclosed herein can optionally comprise agitating the liquid bath to cause the trapped air to escape from the dicot seeds. It is contemplated that optional steps of cleaning and/or sterilizing the dicot seed such as those as disclosed herein can address the issues of excess material or other impurities. In other exemplary aspects, it is contemplated that a dicot seed can be positioned in the selected orientation using conventional means, including, for example and without limitation, at least one of machine visualization with robotic sorting and manipulation, vibratory orientation and feeding, roller sorting and feeding, and manual orientation.

It is contemplated that an opening or hole in the proximal end of the dicot seed can be formed by any conventional means, such as, for example, conventional cutting means. In exemplary aspects, an opening or hole in the proximal end of the dicot seed can be formed by a laser, such as, for example and without limitation, a cold cutting laser, a Q-switched CO₂ laser, a femtosecond laser, a picosecond laser, and a nanosecond laser as are known in the art. In other aspects, an opening or hole in the proximal end of the dicot seed can be formed by a conventional scissors or a knife blade. Optionally, in further exemplary aspects, the proximal end of the dicot seed can be cut at a location between the tip cap (most proximal tip) of the dicot seed and the dicot embryo within the seed to form the hole. In these aspects, the proximal end of the dicot seed can be cut at a location spaced a selected distance from the tip cap to form an opening or hole. It is contemplated that the selected distance from the tip cap can optionally range from about 0.1 mm to about 3 mm. It is further contemplated that the selected distance from the tip cap can optionally range from about 0.25 mm to about 1.5 mm.

Optionally, in exemplary aspects, it is contemplated that the step of forming the hole in the proximal end of the dicot seed can be an iterative process in which multiple cuts are made until a desired location for the hole is achieved. In these aspects, it is contemplated that a first cut can be made proximate the tip cap of the dicot seed. If the location of the first cut is not a desired location for an opening or hole as disclosed herein, then a second cut can be made at a second location positioned closer to the dicot embryo. It is contemplated that this process can be repeated as needed with subsequent cuts until a desired location for an opening or hole is reached.

In additional aspects, a portion of the immature cotyledon section of the dicot embryo can be cut to extract a sample portion of the immature cotyledon section. Optionally, in one aspect, the immature cotyledon tissue can be cut by a laser, such as, for example and without limitation, a cold cutting laser, a Q-switched CO₂ laser, a femtosecond laser, a picosecond laser, and a nanosecond laser as are known in the art. However, it is contemplated that any known means for immature cotyledon removal can be used in conjunction with the systems and methods disclosed herein.

In further aspects, the sample portion of the immature cotyledon section of the dicot embryo can be analyzed. In these aspects, the analysis can comprise at least one of genetic, chemical, and spectral analysis of the sample portion. In additional aspects, the method can further comprise selecting or discarding the remaining portions of the dicot embryo on the basis of the analysis (e.g., genetic analysis) of the sample portion of the immature cotyledon section. When the the dicot embryo is selected on the basis of the analysis (e.g., genetic analysis) of the sample portion of the immature cotyledon section, it is contemplated that the method can further comprise germinating the remaining portions of the dicot embryo (including the meristematic section of the dicot embryo).

Following extraction of the dicot embryo from the dicot seed, the method can further comprise collecting the dicot embryo. It is contemplated that the dicot embryo can be collected by mechanical means. In exemplary aspects, the dicot embryo can be extracted such that the extracted dicot embryo is received within a liquid bath as disclosed herein. In these aspects, it is contemplated that the dicot embryo can be collected through a tube positioned in fluid communication with the liquid bath. In these aspects, it is contemplated that the tube can be configured to sequentially receive individual dicot embryos and transport the dicot embryos to at least one selected receptacle. Optionally, it is contemplated that each sequential dicot embryo can be transported to and received within its own respective receptacle. Alternatively, it is contemplated that a plurality of dicot embryos can be sequentially delivered into a single receptacle. In additional exemplary aspects, it is contemplated that the tube and the at least one selected receptacle can be operatively coupled to a positive pressure source or a negative pressure source, such as, for example and without limitation, a suction pump as is known in the art.

In exemplary aspects, the liquid bath can optionally be filled with at least one of water, solution, buffer, and liquid gel.

In further exemplary aspects, the immature dicot seed can optionally be an immature sunflower seed obtained between 7 and 13 days after pollination.

III. Methods of Automatically Extracting Dicot Embryos from Immature Dicot Seeds

In various aspects, a method is provided for automatically extracting a dicot embryo from an immature dicot seed such that the dicot embryo is undamaged following extraction. The immature dicot seed can have a proximal end and an opposed distal end. Prior to removal of the immature dicot from a biological carrier, the proximal end of the immature dicot seed can be attached to the biological carrier and the distal end of the immature dicot seed can be spaced from the biological carrier.

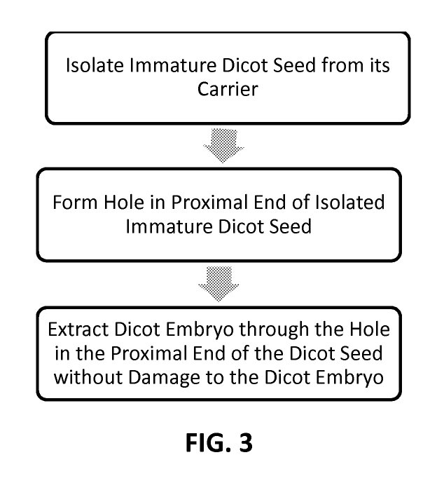

In one aspect, and with reference to FIG. 3, the method can comprise providing an isolated immature dicot seed. Optionally, in an exemplary aspect, the immature dicot seed can have an opening or hole in the proximal end of the dicot seed. In this aspect, the isolated immature dicot seed can be provided following removal of the immature dicot seed from the biological carrier. In another aspect, when the proximal end of the dicot seed has an opening or hole as described herein, the method can comprise automatically extracting the dicot embryo through an opening or hole in the proximal end of the dicot seed. Optionally, it is contemplated that the dicot embryo of the dicot seed can be extracted such that the other material of the dicot seed is not extracted. When the dicot seed has an opening or hole as described herein, it is contemplated that the dicot embryo of the dicot seed can be extracted such that the other material of the dicot seed is not extracted through an opening or hole of the dicot seed. It is still further contemplated that the extracted dicot embryo of the dicot seed can be sufficiently separated from other materials following extraction such that sieving of the extracted dicot embryo is not required. In additional aspects, it is further contemplated that the step of providing the dicot seed can comprise automatically removing at least a portion of the proximal end of the dicot seed to form an opening or hole without damage to the dicot embryo. Optionally, in some aspects, it is contemplated that the dicot embryo can be extracted within a liquid bath as further disclosed herein. In these aspects, it is further contemplated that the extracted dicot embryo can be transported to a selected receptacle as further disclosed herein.

In exemplary aspects, it is contemplated that the immature dicot seed can be an immature sunflower seed.

In an additional aspect, it is contemplated that the step of automatically extracting the dicot embryo can comprise automatically applying pressure to at least one side portion of the dicot seed to force the dicot embryo through an opening or hole in the proximal end of the dicot seed. Alternatively, when an opening or hole is not formed in the dicot seed, it is contemplated that the step of automatically extracting the dicot embryo can comprise automatically applying pressure to at least one side portion of the dicot seed to split a portion of the proximal end of the dicot seed and to force the dicot embryo through an opening or hole defined by the split portion of the dicot seed. In a further aspect, it is contemplated that the method can further comprise forming an opening or hole in the proximal end of the dicot seed. In this aspect, it is contemplated that an opening or hole can optionally be made automatically in the proximal end of the dicot seed. As further described herein, it is contemplated that the step of forming an opening or hole within the proximal end of the dicot seed can be an iterative process. Optionally, in further exemplary aspects, the proximal end of the dicot seed can be cut at a location between the tip cap (most proximal tip) of the dicot seed and the dicot embryo within the seed to form an opening or hole. In these aspects, the proximal end of the dicot seed can be cut at a location spaced a selected distance from the tip cap to form an opening or hole. It is contemplated that the selected distance from the tip cap can optionally range from about 0.1 mm to about 3 mm. It is further contemplated that the selected distance from the tip cap can optionally range from about 0.25 mm to about 1.5 mm. In various exemplary aspects, it is contemplated that the method can further comprise using an imaging system to automatically image the dicot embryo to permit identification of the immature cotyledon and meristematic sections of the extracted dicot embryo.

Optionally, in some aspects, the isolated immature dicot seed can be cleaned and/or sterilized before it is provided. Alternatively, in other optional aspects, it is contemplated that the method can further comprise cleaning at least the proximal end of the dicot seed prior to making the opening in the proximal end of the dicot seed and/or applying pressure to the at least one side portion of the dicot seed. It is further contemplated that the method can comprise sterilizing the dicot seed prior to forming an opening or hole in the proximal end of the dicot seed and/or applying pressure to the at least one side portion of the dicot seed. In exemplary aspects, the method can optionally comprise positioning the dicot seed in a selected orientation as further disclosed herein, such as, for example, prior to forming an opening or hole in the dicot seed and/or prior to extracting the dicot embryo. In these aspects, the step of positioning the dicot seed in a selected orientation can optionally comprise placing the dicot seed in a liquid bath as further disclosed herein.

Optionally, in another aspect, the extracted dicot embryo can be released from an opening or hole of the dicot seed into a liquid bath. In this aspect, the liquid bath can optionally be filled with at least one of water, solution, buffer, and liquid gel. Optionally, in some aspects, it is contemplated that the steps of forming an opening or hole in the dicot seed and/or extracting the dicot embryo from the dicot seed can occur with the dicot seed at least partially submerged within a liquid bath as further disclosed herein.

In a further optional aspect, the method can further comprise automatically removing a sample portion of the immature cotyledon tissue of the extracted dicot embryo without damage to the extracted dicot embryo. Optionally, it is further contemplated that the sample portion of the immature cotyledon tissue can be removed by laser, such as, for example and without limitation, a cold cutting laser, a Q-switched CO₂ laser, a femtosecond laser, a picosecond laser, and a nanosecond laser as are known in the art. However, it is contemplated that any known means for immature cotyledon removal can be used in conjunction with the systems and methods disclosed herein.

In aspects, the sample portion of the immature cotyledon tissue can be analyzed. In these aspects, the analysis can comprise at least one of genetic, chemical, and spectral analysis of the sample portion. In additional aspects, the method can further comprise selecting or discarding the meristematic tissue of the dicot embryo on the basis of the analysis (e.g., genetic analysis) of the sample portion of the immature cotyledon tissue. When the meristematic tissue of the dicot embryo is selected on the basis of the molecular analysis (e.g., genetic analysis) of the sample portion of the immature cotyledon tissue, it is contemplated that the method can further comprise germinating the remaining portion of the dicot embryo comprising the selected meristematic tissue.

In exemplary aspects, an immature dicot seed can optionally be an immature sunflower seed obtained between 7 and 13 days after pollination. An immature seed may be a dicot seed harvested after fertilization but prior to physiological maturity and, except in the case of hard coat seeds, does not germinate in water without external nutrients. For example, harvest may occur between 200 and 750 Growing Degree Days after pollination.

IV. Methods of Regenerating Plants from Immature Dicot Seeds

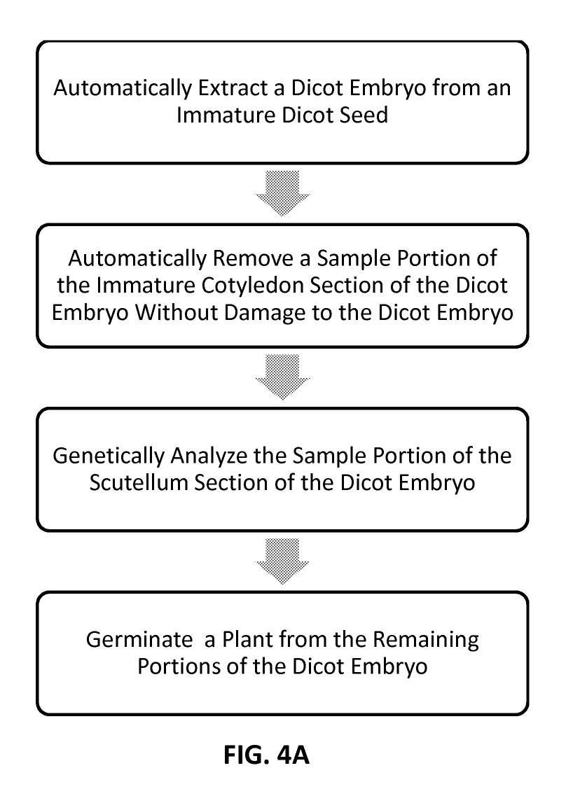

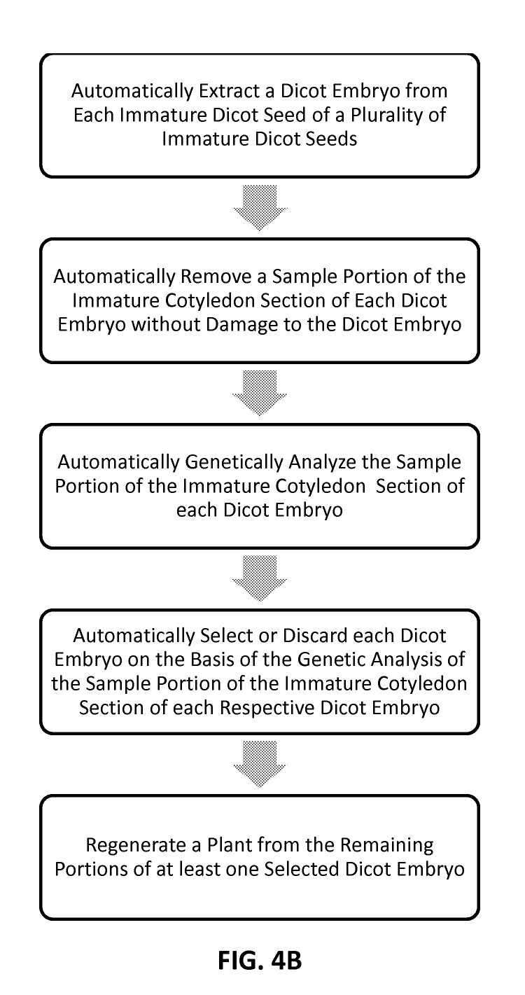

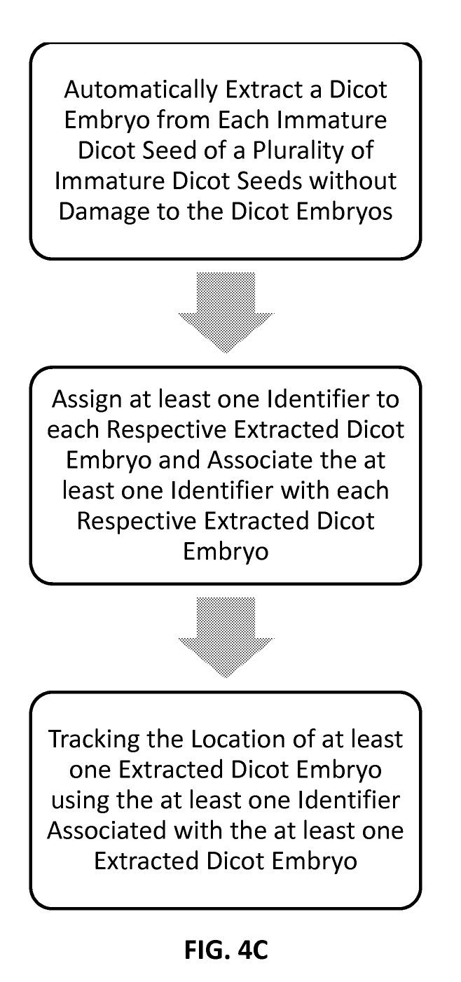

In various aspects, and with reference to FIG. 4A, a method of regenerating a plant from an immature dicot seed is provided. In these aspects, the method can comprise automatically extracting a dicot embryo from the immature dicot seed. As further disclosed herein, the dicot embryo comprises a meristematic section and an immature cotyledon section. It is contemplated that the dicot embryo can optionally be extracted without damaging the dicot embryo. It is further contemplated that the dicot embryo of the dicot seed can be automatically extracted such that most of the other material of the dicot seed is not extracted. It is still further contemplated that the extracted dicot embryo of the dicot seed can be sufficiently separated from other materials following extraction such that sieving of the extracted dicot embryo is not required.

Optionally, in some aspects, it is contemplated that a method can comprise the step of forming an opening or hole in the dicot seed prior to extracting the dicot embryo, as further disclosed herein. As used herein, the terms “opening” and “hole” are used interchangeably and refer to any opening, hole, aperture, or tear through the seed pericarp that exposes a portion of the interior of the seed to the external environment. As further described herein, it is contemplated that the step of forming an opening or hole within the proximal end of the dicot seed can be an iterative process. Optionally, in exemplary aspects, the proximal end of the dicot seed can be cut at a location between the tip cap (most proximal tip) of the dicot seed and the dicot embryo within the seed to form the opening or hole. In these aspects, the proximal end of the dicot seed can be cut at a location spaced a selected distance from the tip cap to form the hole. It is contemplated that the selected distance from the tip cap can optionally range from about 0.1 mm to about 3 mm. It is contemplated that the selected distance from the tip cap can optionally range from about 0.25 mm to about 1.5 mm. In an aspect, it is contemplated that the immature dicot seed can have a proximal end and a distal end, with the proximal end being closer to the dicot embryo than the distal end. In an aspect, it is contemplated that an opening or hole in the proximal end of the immature dicot seed can be formed without damage to the dicot embryo. In exemplary aspects and as further disclosed herein, it is contemplated that an opening or hole in the proximal end of the immature dicot seed can be formed using conventional means, such as, for example and without limitation, scissors, a cutting blade, a laser, and the like. Although described above as being formed by cutting the immature dicot seed, it is contemplated that the opening or hole can also be formed by the application of pressure to and/or within the seed as further described herein. For example, it is contemplated that the opening or hole can be formed at a weakened portion of the proximal end of the seed. In exemplary aspects, the weakened portion of the proximal end of the seed can be formed by chemical degrading a portion of the proximal end. After the weakened portion has been formed, it is contemplated that the opening or hole can be formed by pinching and tearing, poking, scraping, and/or further chemically degrading the weakened portion of the proximal end of the seed.

In exemplary aspects, a method can optionally comprise positioning the dicot seed in a selected orientation as further disclosed herein, such as, for example, prior to forming the hole in the dicot seed and/or prior to extracting the dicot embryo. Optionally, in some aspects, the step of positioning the dicot seed in the selected orientation can comprise placing the dicot seed within a liquid bath as further disclosed herein. In these aspects, the liquid bath can optionally be filled with at least one of water, solution, buffer, and liquid gel. In these aspects, it is contemplated that the step of automatically extracting the dicot embryo from the dicot seed can optionally occur with the dicot seed at least partially submerged within a liquid bath as further disclosed herein.

In still further exemplary aspects, it is contemplated that at least a portion of the dicot seed can be cleaned and/or sterilized as further disclosed herein.

In additional aspects, it is contemplated that the extracted dicot embryo can be transported to a selected receptacle as further disclosed herein.

In another aspect, the method can comprise automatically removing a sample portion of the immature cotyledon section of the dicot embryo without damage to the dicot embryo. In other exemplary aspects, it is further contemplated that the sample portion of the immature cotyledon tissue can be removed by a laser, such as, for example and without limitation, a cold cutting laser, a Q-switched CO₂ laser, a femtosecond laser, a picosecond laser, and a nanosecond laser as are known in the art. However, it is contemplated that any known means for immature cotyledon removal can be used in conjunction with the systems and methods disclosed herein. For example, in some aspects, it is contemplated that the sample portion of the immature cotyledon tissue can be removed by poking, scraping, and/or sloughing as are known in the art. In various exemplary aspects, it is contemplated that the method can further comprise using an imaging system to automatically image the dicot embryo to permit identification of the immature cotyledon and meristematic sections of the extracted dicot embryo.

In still another aspect, the method can comprise analyzing (e.g., genetically analyzing) the sample portion of the immature cotyledon section of the dicot embryo. In this aspect, it is contemplated that the analysis of the sample portion can comprise at least one of genetic, chemical, and spectral analysis. In a further aspect, the method can comprise regenerating a plant from the remaining portions of the dicot embryo (including the meristematic section of the dicot embryo). In this aspect, it is contemplated that the dicot embryo can be undamaged following extraction.

In further aspects, it is contemplated that the step of automatically extracting the dicot embryo can comprise automatically applying force to extract the dicot embryo. In these aspects, it is further contemplated that the step of applying force to extract the dicot embryo can comprise applying pressure to at least one side portion of the dicot seed to force the dicot embryo through the hole in the proximal end of the dicot seed.

In aspects, an immature dicot seed includes but is not limited to, an immature sunflower seed.

In aspects, the immature dicot seed can optionally be an immature sunflower seed obtained between 7 and 13 days after pollination.

It is contemplated that a method of regenerating a plant from selected meristematic tissue of an immature dicot seed can be modified to permit analysis of a plurality of immature dicot seeds. In exemplary aspects, and as shown in FIG. 4B, such a method can comprise automatically extracting a dicot embryo from each respective dicot seed of a plurality of immature dicot seeds. In these aspects, the method can further comprise automatically removing a sample portion of the immature cotyledon section of the dicot embryo of each respective dicot seed without damage to the dicot embryo. Optionally, the dicot embryo of each respective dicot seed can be extracted such that the other material of the dicot seed is not extracted through the hole of the proximal end of the dicot seed. The method can further comprise automatically analyzing (e.g., genetically, chemically, and/or spectrally analyzing) the sample portion of the immature cotyledon section of the dicot embryo of each respective dicot seed. The method can still further comprise automatically selecting or discarding the remaining portions of the dicot embryo of each respective dicot seed on the basis of the analysis of the sample portion of the immature cotyledon section of the dicot embryo of each respective dicot seed. In exemplary aspects, the meristematic tissue of the dicot embryo of at least one dicot seed of the plurality of dicot seeds can be selected. In further aspects, the method can comprise regenerating a plant from the remaining portions of each respective dicot embryo (including the meristematic section).

Optionally, in exemplary aspects, it is contemplated that at least one of the following steps can be performed in an automatic manner: removing a sample portion of the immature cotyledon section of the dicot embryo of each respective dicot seed; genetically analyzing the sample portion of the immature cotyledon section of the dicot embryo of each respective dicot seed; selecting or discarding remaining portions of the dicot embryo of each respective dicot seed on the basis of the genetic analysis of the sample portion of the immature cotyledon section of the dicot embryo of each respective dicot seed; and regenerating a plant from the remaining portions of the dicot embryo of each respective selected dicot seed.

### V. Exemplary Uses of the Extracted Dicot Embryos

In various aspects, and with reference to FIG. 4C, it is contemplated the the dicot embryo extraction methods and systems disclosed herein can permit one-to-one tracking of individual dicot embryos by a large-scale tracking system. In these aspects, it is contemplated that the receptacle containing each respective dicot embryo (or group of dicot embryos from a selected batch) can be assigned at least one unique identifier (e.g., a barcoded batch identification number) that is entered into a database and tracked using conventional automated and/or computerized methods. It is further contemplated that each unique receptacle identifier can be linked with information about the one or more dicot embryos contained within the receptacle, such as, for example and without limitation, the variety of the dicot seed, the specific location (within the field) of the plant from which the dicot embryo was obtained, the location of the dicot seed on its biological carrier (e.g., the location of a sunflower seed on a seed head), and the like. Thus, it is contemplated that each receptacle can be assigned at least one unique identifier that is read and entered into a database of the tracking system. Similarly, it is contemplated that a given batch of dicot embryos can be assigned a unique batch identifier that can be scanned by the tracking system before the extraction process begins. When one or more dicot embryos from a given batch are received within the receptacle, the database entry associated with the receptacle can be updated automatically to associate the receptacle with the batch identifier. Tracking may also comprise creating and using the same or a different unique identifier to associate the embryo of a seed with other portions of the embryo or seed, such as associating the embryo with a sample of that embryo or a sample of the seed from which the embryo was extracted.

Some exemplary applications for the extracted dicot embryos are disclosed below.

a. Plant Breeding

In exemplary aspects, it is contemplated that a dicot embryo extracted as disclosed herein can be used in plant breeding as is known in the art. Generally, in aspects, it is contemplated that a plant can be regenerated from an undamaged dicot embryo extracted as disclosed herein. It is further contemplated that the regeneration of a plant using an extracted dicot embryo can be accomplished using conventional plant breeding methods.

In aspects, it is contemplated that a dicot embryo extracted as disclosed herein can be used for plant breeding purposes as are known in the art, for example for embryos comprising particular traits or genes, whether wild-type or transgenic. In aspects, one or more extracted dicot embryos can be placed in a growth medium with one or more selective agent to assess the resistance of the dicot embryos to one or more selective agents. It is contemplated that dicot embryos with resistance to one or more selective agents will grow while the dicot embryos with insufficient resistance to one or more selective agents will die.

In aspects, it is contemplated that a sample of the immature cotyledon section of an extracted dicot embryo can be obtained before placement of the dicot embryo in a selective or growth medium. In aspects, it is contemplated that a variety of techniques can be used to remove the sample portion of the immature cotyledon section of the dicot embryo. In exemplary aspects, it is contemplated that the process of removing the sample portion of the immature cotyledon section can comprise identifying the orientation and location of an extracted dicot embryo by automated detection means, such as, for example and without limitation, machine vision, imaging systems, and sensing means as are known in the art. The process of removing the sample can further comprise picking up the dicot embryo using automated means (e.g., robotic means). The dicot embryo can then be aligned with a cutting device, such as, for example and without limitation, a tissue-cutting laser. The cutting device can be applied to the immature cotyledon section of the dicot embryo to retrieve the sample in a manner that preserves the viability and vigor of the dicot embryo as disclosed herein. The sample can then be received within a selected receptacle, such as, for example and without limitation, a bullet tube, a field plate, and the like. Following removal of the sample, the process can further comprise positioning the dicot embryo onto a growth medium, such as, for example and without limitation, a tissue culture vessel or other container as is known in the art. Following positioning of the dicot embryo into or onto the selective or growth medium, it is contemplated that the resulting cultured tissue can be used in plant breeding applications as are known in the art.

In exemplary aspects, it is contemplated that a dicot embryo extracted as disclosed herein can be used in a double haploid process as is known in the art. In these aspects, it is contemplated that the double haploid process can optionally be used in conjunction with plant breeding process as are known in the art. —In some aspects, one or more extracted dicot embryos can be placed in a media containing an antimitoticor chromosome doubling agent (e.g., colchicine, oryzalin, amiprophosmethyle or trifluralin) as is known in the art. In these aspects, it is contemplated that the placement of the extracted dicot embryo in the doubling media can cause the doubling of the chromosomes of each dicot embryo. It is further contemplated that the extracted dicot embryos could be selected based upon characteristics, e.g. characterstics revealed through chemical and/or spectral analysis. In additional aspects, after identification of the doubled haploids, the doubled haploids can be placed onto a growth medium, and germination can begin. Optionally, it is contemplated that a sample of the immature cotyledon section of an extracted dicot embryo can be obtained before placement of the dicot embryo in the growth medium.

VI. Exemplary Systems for Extracting and/or Isolating Dicot Embryos

Various systems are contemplated for performing one or more of the method steps disclosed herein.

a. Singulation Assembly

In an exemplary aspect, it is contemplated that dicot seeds can be singulated from other dicot seeds by first positioning the dicot seeds in a liquid bath. In this aspect, the liquid bath can be positioned within a container, and the container can define an outlet opening or passageway configured to receive a single dicot seed at a given time. The container can be configured to promote flow of liquid through the outlet opening or passageway such that an individual dicot seed passes through the opening or passageway, thereby ensuring singulation of each respective dicot seed from the other dicot seeds within the container. Optionally, mechanical means can be provided for effecting movement of the dicot seeds toward the outlet opening or passageway. The outlet opening or passageway can be positioned in fluid communication with a cutting and/or extraction assembly as further disclosed herein.

In other exemplary aspects, it is contemplated that any conventional means for singulating dicot seeds from other dicot seeds can be employed. For example, it is contemplated that the means for singulating dicot seeds can comprise a hopper that is configured to permit exit of only a single dicot seed at a time.

b. Automated Extraction Systems

It is contemplated that any conventional cutting means can be employed to form an opening or hole in the proximal end of a dicot seed as described herein. In exemplary aspects, such a cutting means can be a laser, such as, for example and without limitation, a cold cutting laser, a Q-switched CO₂ laser, a femtosecond laser, a picosecond laser, and a nanosecond laser as are known in the art. In other exemplary aspects, it is contemplated that the cutting means can be a knife or blade assembly as is known in the art. In various aspects, it is contemplated that the cutting means can be incorporated into an automated system for forming an opening or hole in at least one dicot seed as disclosed herein. In aspects, the automated system can be further configured to apply force to the dicot seeds to extract the dicot embryos of the dicot seeds without damage to the dicot embryos.

i. Automated Extraction System Having “Carousel” Assembly

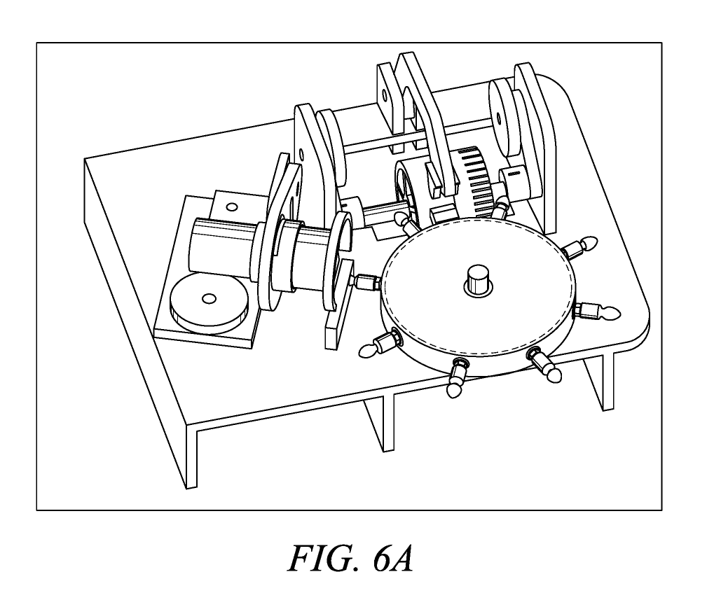

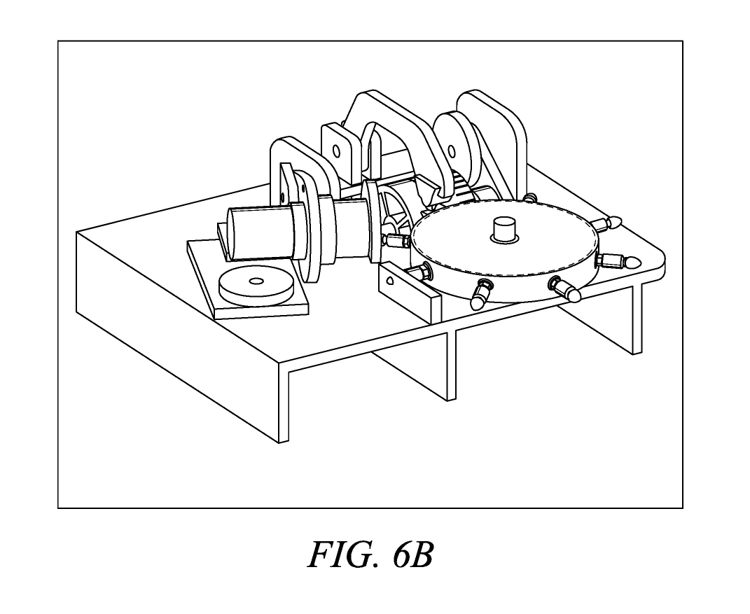

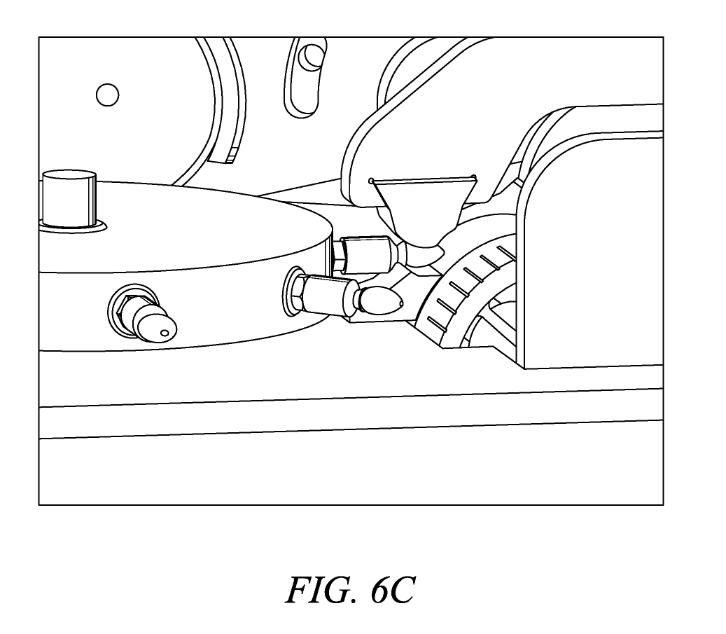

In an exemplary aspect, and with reference to FIGS. 6A-6C, it is contemplated that a dicot embryo extraction system can comprise a carousel subassembly configured for rotation about a rotation axis. In this aspect, the carousel subassembly can comprise a plurality of suction heads circumferentially spaced about the carousel subassembly and sized to respectively engage the distal ends (crowns) of a plurality of dicot seeds. It is contemplated that the suction heads can be positioned in operative communication with a negative pressure source to thereby permit secure engagement between the dicot seeds and the suction heads. Upon engagement between the dicot seeds and the suction heads, it is contemplated that the dicot seeds can be spaced a selected radial distance from the rotation axis. Optionally, the suction heads can project outwardly from the carousel subassembly.

In an aspect, the dicot embryo extraction system can comprise a cutting subassembly having cutting means. In this aspect, the cutting means can be radially positioned relative to the rotation axis such that the cutting means is configured to apply a cutting force to each respective dicot seed at a selected location proximate the proximal end of the dicot seed, such as, for example and without limitation, between about 0.5 mm and about 5 mm from the proximal tip (tip cap) of the dicot seed. Thus, as the carousel subassembly rotates relative to the rotation axis, an opening can be formed in the proximal end of each dicot seed that is engaged with the carousel subassembly. In exemplary aspects, the cutting means can comprise a circular knife blade that is positioned within a plane substantially parallel to the rotation axis and configured for selective rotation. In these aspects, it is contemplated that a fixed support element can be positioned underneath the circular knife blade within the plane of the knife blade to thereby stabilize a dicot seed as the knife blade applies a cutting force to the dicot seed. Optionally, in some aspects, the cutting subassembly can comprise an imaging system positioned in operative communication with the cutting means and configured to optically sense and/or measure the location of the dicot seed. In these aspects, it is further contemplated that the imaging system can be configured to effect adjustment of the position of the cutting means in response to the sensing and/or measurement of the dicot seed.

It is contemplated that the dicot embryo extraction system can comprise an extraction subassembly configured to apply a force to at least one side portion of each respective dicot seed following formation of an opening or hole in the proximal end of the dicot seed by the cutting means. In this aspect, the extraction subassembly can comprise a force application element configured for movement within a plane substantially parallel to the rotation axis. It is contemplated that the force application element can be radially positioned relative to the rotation axis such that the plane of movement of the force application element can intersect a portion of the distal end of each respective dicot seed during rotation of the carousel subassembly, thereby permitting application of force to each dicot seed such that the dicot embryo of the dicot seed is extracted without damage to the dicot embryo. In exemplary aspects, the extraction subassembly can comprise a fixed support element positioned within the plane of movement of the force application element to thereby stabilize a dicot seed as the force application element applies a force to the distal end of the dicot seed. Optionally, the force application element can be a lever-action tool as is known in the art.

In still further exemplary aspects, the dicot embryo extraction system can further comprise a dicot embryo collection subassembly positioned in operative communication with the extraction subassembly. Optionally, it is contemplated that the collection subassembly can comprise a textured roller configured for rotation relative to an axis substantially perpendicular to the rotation axis of the carousel subassembly. It is further contemplated that the roller can be configured to receive dicot embryos extracted from the dicot seeds and rotate such that the dicot embryos are transported to a water bath as further described herein.

ii. Automated Extraction System Having “Vertical Wheel” Assembly

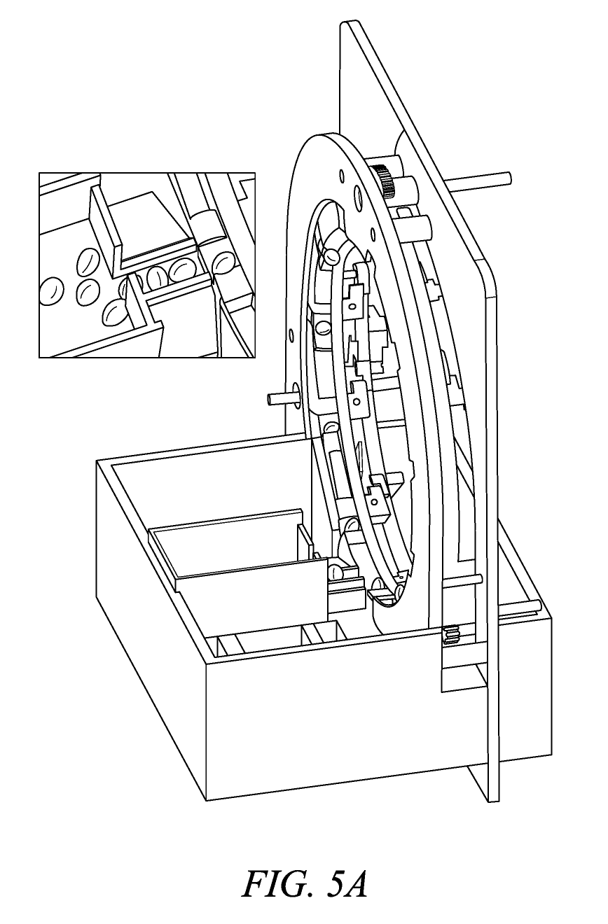

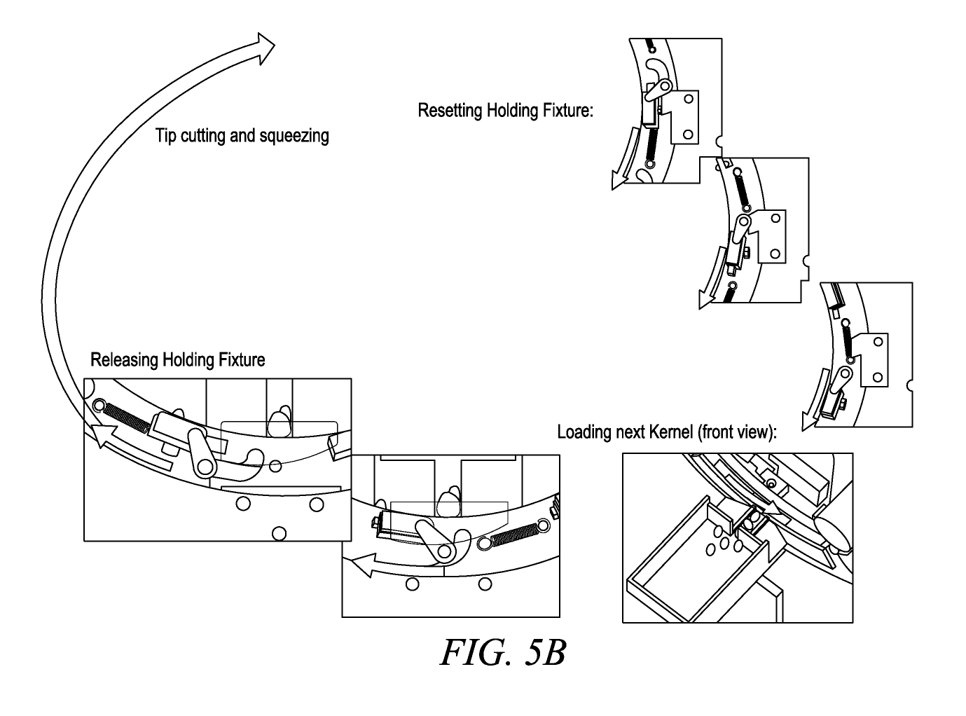

In an exemplary aspect, and as shown in FIGS. 5A-5B, a dicot embryo extraction system can comprise a wheel assembly (such as, for example and without limitation, a vertically oriented wheel assembly) configured to rotate about a rotation axis. In this aspect, it is contemplated that the rotation axis can be oriented substantially parallel to a flat surface supporting the dicot embryo extraction system. It is further contemplated that the wheel assembly can define a plurality of receptacles positioned circumferentially about an inner diameter of the wheel assembly, with each receptacle comprising a dicot seed holding fixture. Each dicot seed holding fixture can be configured to receive and securely engage a respective dicot seed as the wheel assembly rotates about the rotation axis. In exemplary aspects, each holding fixture can be moveable between open and engaged positions, wherein, in the open position, the holding fixture can be configured to receive a dicot seed and in the engaged position, the holding fixture securely engages the dicot seed. As shown, each holding fixture can comprise a pair of spaced gripping elements that are spaced apart a first distance in the open position and spaced apart a second (lesser) distance in the engaged position. It is contemplated that the holding fixtures (and the gripping elements of the holding fixtures) can be spring-loaded, thereby maintaining the orientation and gripping force of the holding fixtures.

In a further aspect, the dicot embryo extraction system can comprise a dicot seed container that is optionally filled with a liquid bath as disclosed herein. In this aspect, the dicot embryo extraction system can permit orientation and/or singulation of the dicot seeds as disclosed herein. The dicot seed container can define an opening or passageway configured to sequentially receive individual dicot seeds positioned within the dicot seed container. It is contemplated that the flow of fluid within the dicot seed container can be selectively controlled to adjust the transport of dicot seeds through the opening or passageway. It is contemplated that the opening or passageway can be positioned in operative communication with the wheel assembly as further disclosed herein.

In exemplary aspects, the opening or passageway of the dicot seed container can be positioned in operative communication with a bottom portion of the wheel assembly such that, when each respective holding fixture is positioned proximate the bottom portion of the wheel assembly, the holding fixture can be configured to receive a respective dicot seed from the dicot seed container. In these aspects, it is contemplated that each holding fixture can be positioned in the open position when it approaches the bottom portion of the wheel assembly. It is further contemplated that the dicot seed container can provide a flow of water that pushes each respective dicot seed into a corresponding holding fixture. In exemplary aspects, the bottom portion can substantially correspond to the six-o'clock position on the wheel assembly.

In further exemplary aspects, it is contemplated that the wheel assembly can be configured for movement in a selected direction (e.g., clockwise or counter-clockwise, as viewed from the front of the wheel assembly). Thus, after a dicot seed is loaded into a holding fixture, upon rotation of the wheel assembly in the selected direction such that the holding fixture passes the bottom portion of the wheel assembly, the holding fixture can be configured to move to the engaged position. In one exemplary aspect, when the wheel assembly is configured for movement in a counter-clockwise direction, it is contemplated that when the holding fixture passes the 5:30 position on the wheel assembly, the holding fixture can be configured to move to the engaged position.

In additional aspects, it is contemplated that the system can be provided with a cutting device positioned at a first selected position on the wheel assembly for forming an opening in the proximal end of a dicot seed as disclosed herein. In one aspect, it is contemplated that the cutting device can optionally be an automated cutting element that is selectively adjustable based upon measurement of the location and/or dimension of a dicot seed by an imaging system as is known in the art. In other optional aspects, it is contemplated that the cutting device can be a mechanically-offset cutter, such as, for example and without limitation, a potato-peeler-style cutter, a mandolin-slicer style cutter, or a cheese grater-style cutter. With the holding fixture positioned in the engaged position, when the holding fixture reaches the first selected position on the wheel assembly, it is contemplated that the cutting device can be activated to form the opening in the proximal end of the dicot seed positioned within the holding fixture. In one exemplary aspect, when the wheel assembly is configured for movement in a counter-clockwise direction, it is contemplated that the first selected position can substantially correspond to a three-o'clock position on the wheel assembly.

In further aspects, it is contemplated that the wheel assembly can be provided with means for applying additional external positive force to at least one side portion of a dicot seed as disclosed herein. In these aspects, the means for applying additional external positive force to the distal end of the dicot seed can be positioned at a second selected position on the wheel assembly. In exemplary aspects, it is contemplated that the means for applying additional external positive force to the distal end of a dicot seed can be configured for operative coupling to the gripping members of each respective holding fixture to further compress the distal end of the dicot seed received within the gripping members. In additional aspects, upon application of the additional force to the dicot seed, it is contemplated that the dicot embryo of the dicot seed can be extracted through the hole in the proximal end of the dicot seed as disclosed herein. It is further contemplated that the system can comprise a roller, belt, trough, or other means for receiving the extracted dicot embryos and transporting the dicot embryos to one or more selected receptacles without damage to the dicot embryos. Thus, in operation, after the hole is formed in a respective dicot seed, when the holding fixture containing the dicot seed reaches the second selected position on the wheel assembly, additional external positive force can be applied to the at least one side portion of the dicot seed to extract the dicot embryo as disclosed herein. It is contemplated that the extracted embryo can be received and automatically transported to a selected receptacle without damage to the dicot embryo. In one exemplary aspect, when the wheel assembly is configured for movement in a counter-clockwise direction, it is contemplated that the second selected position can substantially correspond to a twelve-o'clock position on the wheel assembly.

In additional aspects, it is contemplated that the system can comprise means for mechanically moving each respective holding fixture to the open position. In these aspects, it is contemplated that the means for mechanically moving each respective holding fixture to the open position can be positioned between the second selected position and the bottom portion of the wheel assembly (relative to the circumference of the wheel assembly). In one exemplary aspect, when the wheel assembly is configured for movement in a counter-clockwise direction, it is contemplated that the means for mechanically moving each respective holding fixture to the open position can be positioned between the twelve-o'clock and six-o'clock positions on the wheel assembly.

In still further aspects, it is contemplated that the system can comprise means for removing remnants of the dicot seed within each respective holding fixture. In these aspects, it is contemplated that the means for removing remnants of the dicot seed can be positioned between the means for mechanically moving each respective holding fixture to the open position and the bottom portion of the wheel assembly (relative to the circumference of the wheel assembly). Exemplary means for removing the remnants of the dicot seed can comprise negative pressure sources and fluid dispensers. It is contemplated that when each respective holding fixture returns to the bottom portion of the wheel assembly, the above process can be repeated for another dicot seed.

As shown, it is contemplated that the wheel assembly can comprise a plurality of interior channels and/or projections that are configured to engage portions of the holding fixtures and effect movement of the holding fixtures between the open and engaged positions as disclosed herein.

iii. Automated Extraction Device

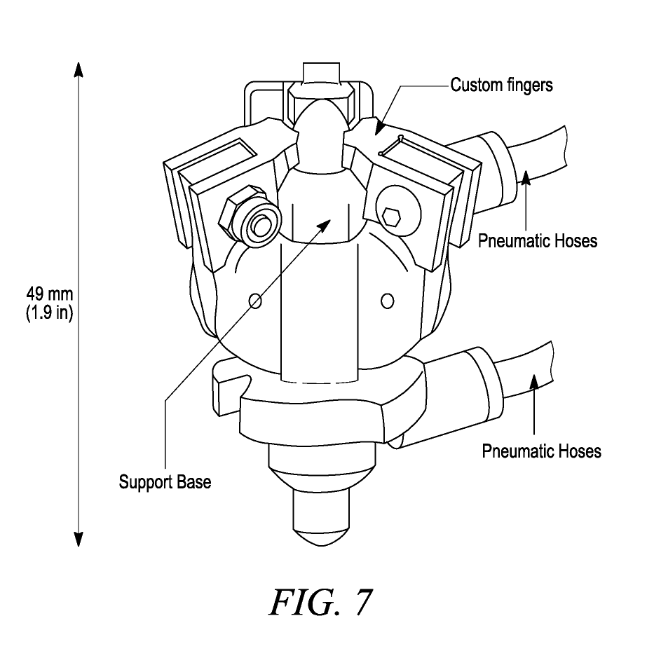

In an exemplary aspect, and as shown in FIG. 7, an automated extraction device for extracting a dicot embryo from a dicot seed is provided. In this aspect, the extraction device can have a support base operatively coupled to a bottom portion of the device. The support base can be configured to support a dicot seed in a desired orientation as disclosed herein. The extraction device can further comprise a plurality of circumferentially spaced fingers that cooperate with the support base to define a central receiving space for a dicot seed. The plurality of circumferentially spaced fingers can be oriented toward a central axis of the device that is substantially aligned with a center point of the dicot seed. The plurality of circumferentially spaced fingers can be configured for selective radial movement between an open position and a gripping position. The plurality of circumferentially spaced fingers can be configured to selectively apply a radial force to the dicot seed. In exemplary aspects, the plurality of fingers can be configured to apply a force of a first magnitude sufficient to support the dicot seed during formation of an opening or hole in the dicot seed as disclosed herein. In these aspects, the plurality of fingers can be further configured to apply a second force of a second magnitude sufficient to force the dicot embryo of the dicot seed through the hole of the dicot seed without damaging the dicot embryo. It is contemplated that the plurality of fingers can optionally be activated using pneumatic means, such as, for example and without limitation, pneumatic hoses. In exemplary aspects, the plurality of fingers can comprise three fingers. Generally, it is contemplated that an odd number of gripping surfaces can provide advantageous centering. It is further contemplated that angling of the fingers as shown can produce an advantageous in-and-down squeezing profile. In exemplary aspects, the plurality of fingers can be configured to apply a radial extraction force in a pulsed manner. It is still further contemplated that gripping surfaces of the fingers can have a curvature that generally matches the curvature of a dicot seed. In further exemplary aspects, it is contemplated that the automated extraction device can comprise a 3-jaw robotic manipulator, with each jaw being coupled to a respective finger and the support base being mounted to a top surface of the robotic manipulator. In still further exemplary aspects, it is contemplated that the automated extraction device can be configured for underwater usage.

c. Dicot Embryo Transport Assembly

Following extraction of a dicot embryo from a dicot seed as disclosed herein, the dicot embryo can be collected from a container, such as, for example and without limitation, a container containing a liquid bath as described herein. It is contemplated that the dicot embryo can be collected by any mechanical means that avoids damage to the dicot embryo. In exemplary aspects, it is contemplated that the dicot embryo can be collected through a tube positioned in fluid communication with the container. In these aspects, it is contemplated that the tube can be configured to sequentially receive individual dicot embryos and transport the dicot embryos to at least one selected receptacle. Optionally, in additional exemplary aspects, it is contemplated that the tube and the at least one selected receptacle can be operatively coupled to a positive pressure source or a negative pressure source, such as, for example and without limitation, a suction pump as is known in the art.

d. Analysis Systems

In exemplary aspects, it is contemplated that the analysis steps of the disclosed methods can be performed using conventional genetic analysis equipment, conventional chemical analysis equipment, and/or conventional spectral analysis equipment, and/or automated systems combining any number of these steps, including, for example and without limitation, extraction, dilution, and the like.

e. Dicot Seed Removal Apparatus

When the immature dicot seed is an immature sunflower seed, it is contemplated that the dicot seed can be provided for use in the disclosed methods following removal of the dicot seed from its seed head. In aspects, it is contemplated that the immature sunflower seeds can be manually removed without rupturing of the dicot seeds. In other exemplary aspects, it is contemplated that the immature sunflower seeds can be removed by an apparatus configured to cut sufficiently close to the seed head to avoid damage to the dicot embryos of the dicot seeds

The following examples are offered by way of illustration and not by way of limitation.

### EXPERIMENTAL

### Example 1

### Comparison of the Condition and Quality of Immature Embryos Extracted Using Five Different Methods

Immature embryos were extracted using five different methods: hand extraction; high-pressure water; suction; hand squeezing; and semi-automated squeezing. The condition and quality of extracted embryos was evaluated for each respective method.

One ear of immature corn was used for each extraction method. The embryos were 2.0-3.5 mm long and were extracted 12-18 days post-pollination (corresponding to 340-380 Growing Degree Units (GDUs)). The embryos were substantially the same size as those used in typical Doubled Haploid production methods.

i. Sterilization and Cleaning

Initially, the husks and silks were removed from four ears of immature corn. After removal of the husks and silks, each ear was secured to an ear holder. The ears were then positioned within an empty pitcher. The pitcher was then filled with a solution containing 20%-50% CLOROX Bleach, one drop of TWEEN® 20, and tap water. The ears were soaked in the sterilizing solution, with the ears being rotated occasionally using the ear holders. After 15-20 minutes of soaking, the ears were removed from solution and then rinsed three times with tap water.

ii. Seed Preparation

For the hand, high-pressure water, and suction extraction methods, the ear was held using the ear holder, and a scalpel was used to slice off the kernel caps of each corn seed on the ear.

For the hand-squeezing extraction method, the ear was held using the ear holder, and a fingertip was used to remove whole seeds from the cob. A scalpel was then used to slice off the individual kernel tips of the seeds.

For the semi-automated squeezing extraction method, the ear was held using the ear holder, and a fingertip was used to remove whole seeds from the cob.

iii. Embryo Extraction

For the hand extraction method, a spatula was used to remove 30 embryos from an ear (with the seed caps removed).

For the high-pressure water extraction method, an ear (with the seed caps removed) was selectively positioned, and a hand-pressure nozzle was used to wash the embryos out of the seeds and into catch pans (dishes). Tap water was used to wash the embryo debris through mesh sieves of decreasing size until the embryos could be clearly identified. A spatula was used to remove 30 embryos from the sieve.

For the vacuum extraction method, a vacuum pump was used to remove embryos from an ear (with the seed caps removed) via tubing. The tubing was positioned in fluid communication with an Erlenmeyer flask such that the extracted embryos were delivered to the flask. The embryos within the flask were washed with tap water through mesh sieves of decreasing size to separate the embryos from embryo debris until the embryos could be clearly identified. A spatula was used to remove 30 embryos from the sieve.

For the hand-squeezing extraction method, each seed (with its tip removed) was positioned between the thumb and forefinger of an operator, and pressure was gently applied until the embryo popped out of the seed into a clean container. A spatula was used to remove 30 embryos from the container.

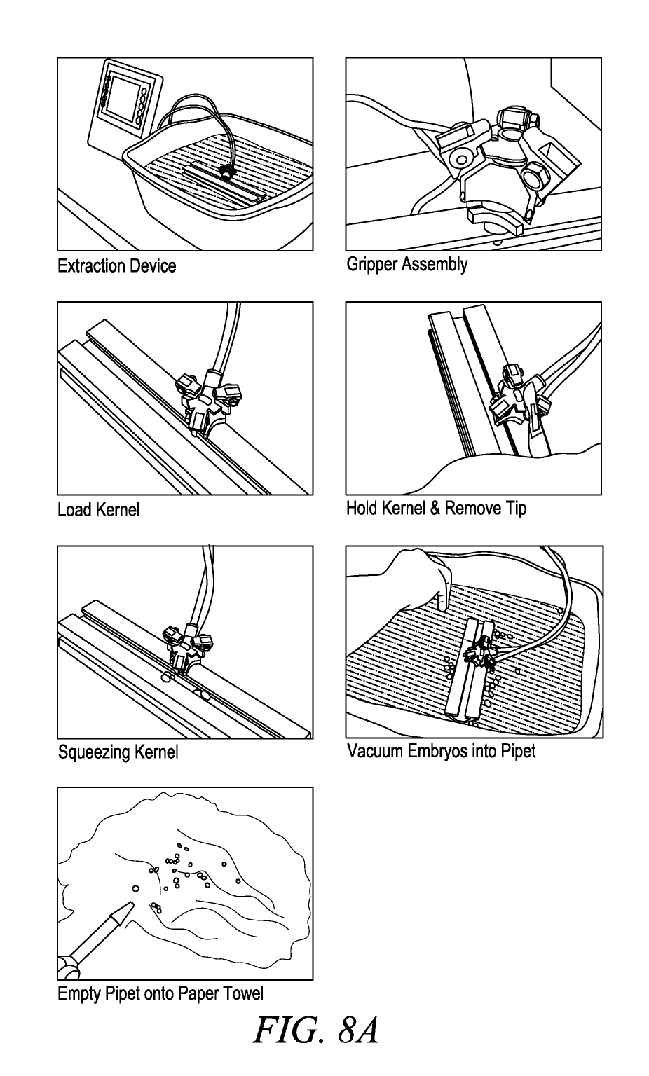

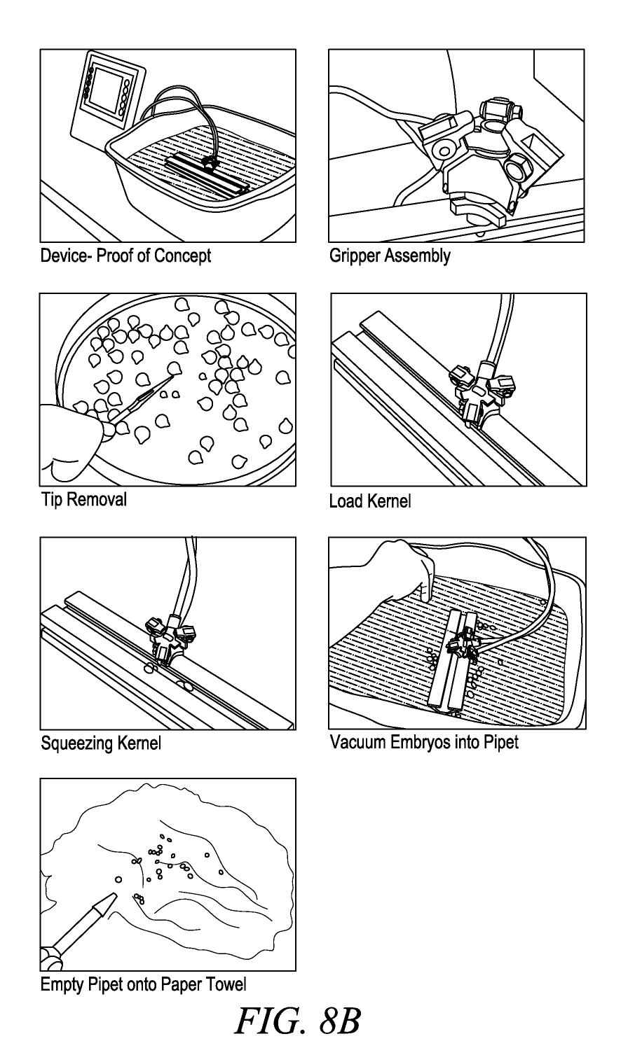

For the semi-automated squeezing extraction method, an automated extraction device as disclosed herein and depicted in FIG. 7 was set with its fingers in an open position. An individual kernel was positioned within the central receiving space defined between the open fingers and the support base of the extraction device. The kernel was positioned with its tip facing up, and the fingers of the extraction device were advanced to a gripping position to hold the kernel in the desired orientation. In a first trial, a scalpel was used to remove a portion of the tips of some kernels (without damaging the embryo) while the kernels were gripped by the fingers of the extraction device, while the tips of other kernels were removed prior to positioning of the kernels within the extraction device. In a second trial, the tips of all kernels were removed prior to positioning of the kernels within the extraction device. With each kernel gripped by the fingers of the extraction device, the fingers were radially advanced to apply an extraction force to the sides of the kernel. The extraction force was pulsed as necessary until the embryo exited the hole formed in the tip of the kernel. The fingers were then returned to the open position, and the extracted embryo was collected. This process was repeated until 30 embryos were extracted and collected. Images depicting the performance of the first trial are provided in FIG. 8A, and images depicting the performance of the second trial are provided in FIG. 8B.

iv. Embryo Plating

Five petri dishes were filled with 605J Resting Media, and one petri dish was filled with 272X Resting Media. With the lid of each petri dish removed, 30 extracted embryos for each respective extraction method were evenly distributed on the resting media (to ensure adequate separation between the embryos) in a corresponding petri dish using a spatula. The embryos extracted using the hand extraction, high-pressure water, suction, and hand-squeezing methods were positioned within the 605J Resting Media, and the embryos extracted using the semi-automated squeezing method were positioned within the 272X Resting Media. The lids were replaced on each petri dish, and a marker was used to label each petri dish in accordance with the method used to extract the embryos within the petri dish and the position of the petri dish relative to the other petri dishes.

v. Embryo Analysis

Each embryo was analyzed under a microscope, and a score of the condition and quality of each embryo was independently recorded by two different analysts. The analysts looked for damage, defects, or other stress issues that may have an adverse effect on the growth and/or vigor of the embryo. Each embryo was rated on a general condition scale of 1-3, with 1=Excellent Condition, 2=Minor Defects or Damage (still capable of normal growth), and 3=Major Defects or Damage (unlikely to grow or grow normally). Any explanations or comments about each embryo's condition or quality were recorded. A digital record of each petri dish and its contents was prepared.

vi. Results

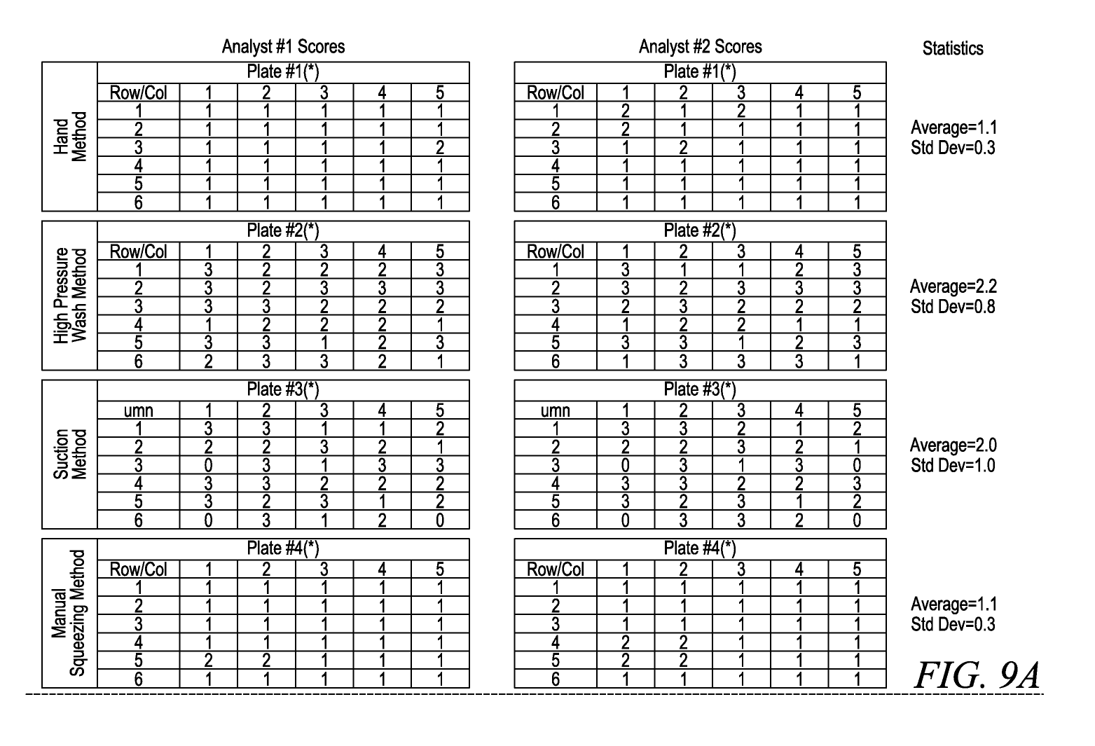

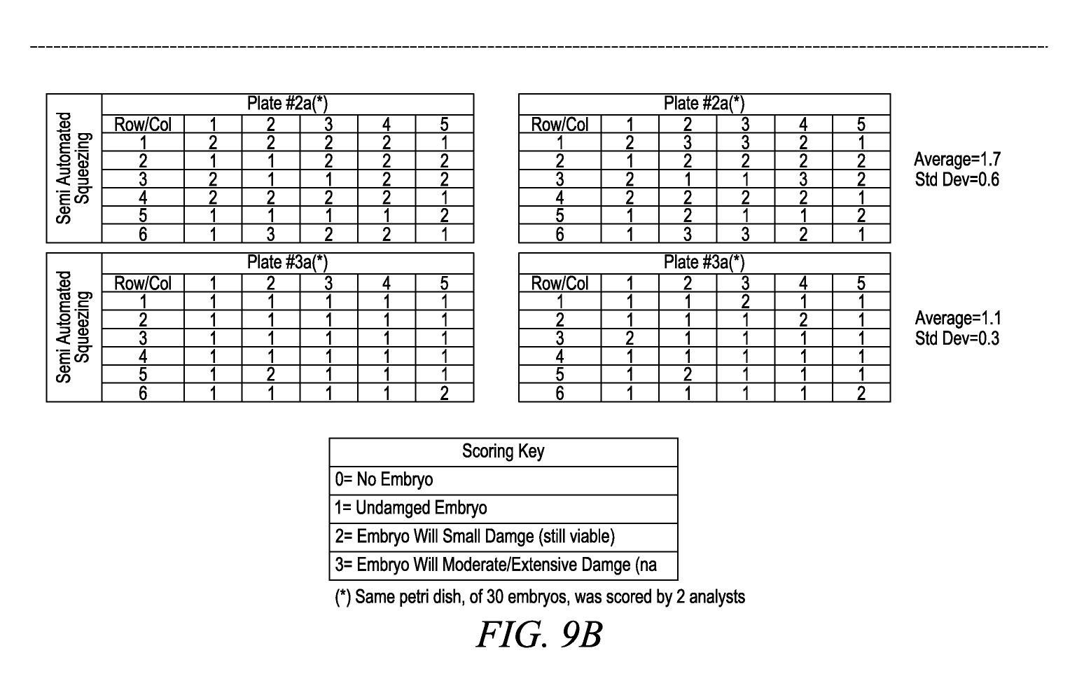

The embryo condition and quality scores are shown in the table provided as FIG. 9. As indicated in the table, the condition and quality of the embryos extracted by manual hand-squeezing and hand methods were very good and substantially the same. The condition and quality of the embryos extracted by the semi-automated extraction method were slightly below that of the embryos extracted by the manual hand-squeezing and hand extraction methods. The condition and quality of the embryos extracted by suction or high-pressure washing methods were poor compared to the hand-squeezing, hand, and semi-automated extraction methods.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

As used in the specification and the appended claims, the singular forms “a,” “an” and “the” include plural referents unless the context clearly dictates otherwise.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. For example, in an aspect, a complex comprising a targeting moiety and an oligonucleotide can optionally comprise a detectable label. In an aspect, a disclosed method can optionally comprise repeating the administration of a disclosed composition and/or complex.

Although the foregoing invention has been described in some detail by way of illustration and example for purposes of clarity of understanding, certain changes and modifications may be practiced within the scope of the appended claims.

1. A method of manually or automatically isolating a dicot embryo from an immature dicot seed having a proximal end and an opposed distal end, wherein, prior to removal of the immature dicot seed from its biological carrier, the proximal end of the immature dicot seed is attached to the biological carrier and the distal end of the immature dicot seed is spaced from the biological carrier, the method comprising:
a. providing an isolated immature dicot seed having an opening or hole in the proximal end of the dicot seed, wherein the isolated immature dicot seed is provided following removal of the immature dicot seed from the biological carrier; and
b. extracting the dicot embryo through the opening or hole in the proximal end of the isolated immature dicot seed, wherein the dicot embryo is undamaged following extraction.

2. The method of claim 1, wherein, following extraction of the dicot embryo, the dicot embryo retains an ability to grow with full viability and vigor.

3. The method of claim 1, wherein the step of providing the dicot seed comprises removing at least a portion of the proximal end of the dicot seed without damage to the dicot embryo.

4. The method of claim 1, wherein the immature dicot seed is a sunflower seed.

5. The method of claim 1, wherein the immature dicot seed comprises side portions extending between its proximal and distal ends, and wherein the step of extracting the dicot embryo through the hole comprises applying pressure to at least one side portion of the dicot seed to force the dicot embryo through the hole in the proximal end of the dicot seed.

6. The method of claim 1, further comprising forming the opening or hole in the proximal end of the dicot seed.

7. The method of claim 1, wherein generally the extracted dicot embryo is released into a liquid bath and generally all other contents of the seed are not released into the liquid bath.

8. The method of claim 1, further comprising automatically collecting the extracted dicot embryo.

9. The method of claim 8, further comprising positioning the extracted dicot embryo within or onto a medium.

10. The method of claim 9, wherein the medium is a growth medium.

11. The method of claim 9, wherein the medium is a medium containing a selective agent.

12. The method of claim 9, wherein the medium comprises an antimitotic or chromosome doubling agent.

13. The method of claim 1, wherein the extracted dicot embryo comprises a immature cotyledon section and a meristematic section, and wherein the method further comprises removing a sample portion of the immature cotyledon section of the extracted dicot embryo.

14. The method of claim 13, wherein the sample portion of the dicot embryo and the remaining viable portion of the dicot embryo are tracked 1 to 1 for future identification and retrieval.

15. The method of claim 13, wherein, following removal of the sample portion of the immature cotyledon section of the extracted dicot embryo, the dicot embryo retains an ability to grow with full viability and vigor.

16. The method of claim 13, further comprising using an imaging or measurement system to characterize the extracted dicot embryo to permit identification of the immature cotyledon and meristematic sections of the extracted dicot embryo

17. The method of claim 13, wherein the sample portion of the immature cotyledon section of the extracted dicot embryo is removed by laser.

18. The method of claim 17, wherein the laser is selected from the group consisting of a cold cutting laser, a Q-switched CO₂ laser, a femtosecond laser, a picosecond laser, and a nanosecond laser.

19. The method of claim 13, further comprising analyzing the sample portion of the immature cotyledon section of the extracted dicot embryo.

20. The method of claim 19, wherein the analyzing comprises genetic analysis of the sample.

21. The method of claim 20, further comprising selecting or discarding remaining portions of the extracted dicot embryo on the basis of the genetic analysis of the sample portion of the immature cotyledon section of the extracted dicot embryo.

22. The method of claim 21, further comprising germinating the remaining viable portions of the selected dicot embryo

23. The method of claim 21, wherein the selected dicot embryo is used in a dicot breeding program.

24. The method of claim 1, wherein the immature dicot seed is a sunflower seed obtained at a predetermined time after pollination.

25. A method of regenerating a plant from an immature dicot seed, the method comprising:
a. automatically extracting a dicot embryo from a dicot seed, the extracted dicot embryo comprising a meristematic section and a immature cotyledon section, wherein the extracted dicot embryo is undamaged following extraction,
b. removing a sample portion of the immature cotyledon section of the dicot embryo without damage to the extracted dicot embryo,
c. identifying and associating the sample to viable embryo portion for tracking in a 1 to 1 relationship;
d. genetically analyzing the sample portion of the immature cotyledon section of the dicot embryo, and
e. generating a plantlet from remaining portions of the extracted dicot embryo, wherein the remaining portions of the extracted dicot embryo comprise the meristematic section of the extracted dicot embryo.

26. The method of claim 25, wherein following extraction of the dicot embryo and removal of the sample portion of the immature cotyledon section of the dicot embryo, the dicot embryo retains an ability to grow with full viability and vigor.

27. The method of claim 25, wherein the immature dicot seed has a proximal end and an opposed distal end, wherein, prior to removal of the immature dicot seed from a biological carrier, the proximal end of the immature dicot seed is attached to the biological carrier and the distal end of the immature dicot seed is spaced from the biological carrier, wherein the dicot embryo is extracted from the dicot seed following removal of the dicot seed from the biological carrier, and wherein an opening or hole is formed in the proximal end of the dicot seed without damage to the dicot embryo.

28. The method of claim 27, wherein the opening or hole is formed in the proximal end of the dicot seed using a cutting blade, pinching and tearing, poking, scraping, chemically degrading, using a a laser or other cutting elements.

29. The method of claim 25, wherein the sample portion of the immature cotyledon section of the dicot embryo is removed by laser, poking, scraping, or sloughing.

30. The method of claim 29, wherein the laser is selected and configured to minimize a heat affected zone of the dicot embryo.

31. A method of extracting an immature sunflower embryo, comprising:
a. Obtaining an isolated immature sunflower seed removed from a seed head;
b. Placing the immature sunflower seed in a liquid bath, wherein upon positioning of the immature sunflower seed in the liquid bath, the immature sunflower seed achieves a desired orientation; and
c. Removing the tip cap of the immature sunflower seed;
d. Applying force to extract the sunflower embryo from the sunflower seed; and.
e. collecting the embryo

32. The method of claim 31, wherein the immature sunflower seed has a proximal end and an opposed distal end, wherein, prior to removal of the immature sunflower seed from the seed head, the proximal end of the immature sunflower seed is attached to the seed head and the distal end of the immature sunflower seed is spaced from the seed head, wherein the immature sunflower seed has an opening or hole in the proximal end before it is placed in the liquid bath in step (b), or an opening or hole in the proximal end is formed after the step of placing the sunflower seed in the liquid bath in step (b).

33. The method of claim 31, wherein, following extraction of the sunflower embryo, the sunflower embryo retains an ability to grow with full viability and vigor.

34. The method of claim 31, wherein a portion of a immature cotyledon section of the sunflower embryo is cut to extract a sample portion of the immature cotyledon section.

35. The method of claim 34, wherein the immature cotyledon section is cut by laser.

36. The method of claim 34, wherein the sample portion of the immature cotyledon section is genetically analyzed.

37. The method of claim 36, further comprising selecting or discarding the sunflower embryo on the basis of an analysis of the sample portion of the immature cotyledon section.

38. The method of claim 37, further comprising germinating remaining portions of the selected dicot embryo.

39. The method of claim 31, wherein the liquid bath is filled with at least one of water, sterile solution, buffer, and liquid gel.

40. The method of claim 31, wherein the immature sunflower seed is obtained at a predetermined time after pollination.

41. A method of regenerating a plant from an immature dicot seed, the method comprising:
a. automatically extracting a dicot embryo from each respective dicot seed of a plurality of dicot seeds, each extracted dicot embryo comprising a meristematic section and a immature cotyledon section, wherein the extracted dicot embryo of each respective dicot seed is undamaged following extraction,
b. removing a sample portion of the immature cotyledon section of the dicot embryo of each respective dicot seed without damage to the dicot embryo and identifying and associating the embryo and the sample for tracking the sample portion and the dicot embryo in a 1 to 1 relationship,
c. genetically analyzing the sample portion of the immature cotyledon section of the dicot embryo of each respective dicot seed, and
d. selecting or discarding remaining portions of the dicot embryo of each respective dicot seed on the basis of the genetic analysis of the sample portion of the immature cotyledon section of the dicot embryo of each respective dicot seed, wherein the remaining portions of the dicot embryo of at least one dicot seed of the plurality of dicot seeds is selected; and
e. germinating a plant from the remaining portions of the dicot embryo of each respective selected dicot seed.

42. The method of claim 41, wherein following extraction of the dicot embryo of each respective dicot seed and removal of the sample portion of the immature cotyledon section of the dicot embryo of each respective dicot seed, the dicot embryo of each respective dicot seed retains an ability to grow with full viability and vigor.

43. The method of claim 41, wherein each respective dicot seed has a proximal end and an opposed distal end, wherein, prior to removal of each immature dicot seed from a biological carrier, the proximal end of the immature dicot seed is attached to the carrier and the distal end of the immature dicot seed is spaced from the carrier, wherein the dicot embryo of each respective immature dicot seed is extracted from the dicot seed following removal of the dicot seed from the carrier, and wherein an opening or hole is formed in the proximal end of each respective dicot seed without damage to the dicot embryo of the dicot seed.

44. The method of claim 41, wherein the method does not comprise sieving material extracted from each dicot seed to separate the dicot embryo of each respective dicot seed from other extracted material.

45. The method of claim 41, further comprising:
a. assigning the extracted dicot embryo of each respective dicot seed at least one identifier, each identifier being indicative of at least one of a variety of the dicot seed from which the dicot embryo was obtained;
b. associating at least one identifier to each respective extracted dicot embryo; and
c. tracking a location of at least one extracted dicot embryo using the at least one identifier associated with the at least one extracted dicot embryo.

46. The method of claim 41, wherein at least one of the steps of (b) removing a sample portion of the immature cotyledon section of the dicot embryo of each respective dicot seed, (c) genetically analyzing the sample portion of the immature cotyledon section of the dicot embryo of each respective dicot seed, (d) selecting or discarding remaining portions of the dicot embryo of each respective dicot seed on the basis of the genetic analysis of the sample portion of the immature cotyledon section of the dicot embryo of each respective dicot seed, and (e) regenerating a plant from the remaining portions of the dicot embryo of each respective selected dicot seed is performed automatically.
